(19) United States
(12) Patent Application Publication
(10) Pub. No.: US 2026/0327537 A1
(43) Pub. Date: Oct. 22, 2026

(54) DEVICE FOR CELL OR TISSUE CULTURE

(71) Applicants: UNIVERSITÉ CLAUDE BERNARD LYON 1, Villeurbanne Cedex (FR); CENTRE NATIONAL DE LA RECHERCHE SCIENTIFIQUE, PARIS (FR); INSTITUT NATIONAL DES SCIENCES APPLIQUEES DE LYON, VILLEURBANNE (FR); ECOLE SUP CHIMIE PHYS ELECTRONIQ LYON, VILLEURBANNE CEDEX (FR)

(72) Inventors: Christophe MARQUETTE, VILLEURBANNE (FR); Hamza Raza ZAIDI, LYON (FR)

(73) Assignees: UNIVERSITÉ CLAUDE BERNARD LYON 1, Villeurbanne Cedex (FR); CENTRE NATIONAL DE LA RECHERCHE SCIENTIFIQUE, PARIS (FR); INSTITUT NATIONAL DES SCIENCES APPLIQUEES DE LYON, VILLEURBANNE (FR); ECOLE SUP CHIMIE PHYS ELECTRONIQ LYON, VILLEURBANNE CEDEX (FR)

(21) Appl. No.: 19/110,560

(22) PCT Filed: Sep. 28, 2023
(86) PCT No.: PCT/EP2023/076847

§ 371 Date: Mar. 11, 2025

(30) Foreign Application Priority Data
Sep. 30, 2022 (FR) 2209949

(51) Int. Cl. C12M 3/00; C12M 1/00

(52) U.S. Cl. CPC C12M 21/08; C12M 23/34; C12M 23/42

(57) ABSTRACT

A cell culture device including an insert having an internal cavity delimited by an insert side wall and an insert bottom, the internal cavity being able to receive the cell culture in a cell culture fluid so as to create a cell tissue. The device includes a container that removably receives the insert, the side wall having first through-holes. The container includes an internal flue and an opening which allows the insert to be inserted into the flue. The insert bottom includes second through-holes allowing observation of the cells therethrough, the first and second through-holes being capillary holes able to retain the cell culture fluid in the internal cavity and serving to anchor the cell tissue.

### FIELD OF THE INVENTION

The invention relates to a device for cell or tissue culture, comprising a container and an insert. The insert is able to be placed in said container and able to receive cells or tissues to be cultured.

Cell culture and tissue culture are considered to be two types of process used to grow cells of multicellular organisms outside their natural environment.

The aim of cell culture is to grow cells outside the organism. Cell culture is widely used: it makes it possible to carry out pharmacological and chromosomal studies, and is a tool in fundamental research, antibody production, protein synthesis, etc.

### PRIOR ART

There is a great need to carry out experiments on cell culture or tissue culture samples in a wide range of fields, in particular cosmetics and/or pharmaceuticals.

For example, there is a great need to study and develop therapies for healthy or diseased skin. These therapies increasingly use skin equivalent models on which the products are tested.

These skin equivalent models are increasingly sought-after, and have been created following the European Union's complete ban on animal testing.

Skin equivalent models are created by culturing cells in devices, and are for example in the form of artificial skin tissue.

The methods used make it possible to create artificial skin tissues. The aim is to create artificial skin tissues, or artificial cell tissues, which have properties similar to those of native human skin, both structurally and functionally. More generally, the aim is to create soft tissues, for example skin tissues, but also liver, kidney, lung, adipose, pancreatic and other tissues.

The use of cell culture inserts is a well-known means for producing artificial skin tissues. These inserts are designed to enable static cell culture, during which skin cells are matured under stress-free environmental conditions, until tissue of the desired size and thickness is obtained.

Document WO2011127945 describes, for example, a cell culture system using inserts which are placed in a container and into which the cells or tissues are introduced in order to be cultured. Each insert makes it possible to create a cell tissue which can then be used for testing cosmetic or pharmaceutical products, for example. In the example shown in document WO2011127945, the inserts each have a side wall and a bottom wall, the side wall having through-openings through which a liquid can pass. These inserts are placed in wells which have been formed in a support. A liquid which enables the cells to be cultured is introduced into the wells.

Cell tissues are obtained after a certain period of time, this being the time required for the tissues or cells to develop under stress-free environmental conditions.

Underlying the invention was the aim of improving the properties of the artificial skin obtained, i.e. improving the properties of the cell tissue so that it is even closer to a natural skin tissue: indeed, to date, the artificial skin tissues created have not really mimicked certain natural mechanical strength properties that natural skin exhibits during its development.

It is known that the natural growth of human skin occurs simultaneously when the human body is exposed to multiple stresses and environmental conditions: it has been observed, from several studies, that when fibroblast cells are exposed to different types of stress, this leads to an increase in collagen production due to the activation of several signaling pathways in the cell in response to the stress to which the surrounding collagen protein is exposed.

The production of collagen proteins provides a certain degree of mechanical strength to the skin's overall structure. The invention aims to achieve these natural mechanical strength properties in the artificial skin tissues created.

Some studies have succeeded in developing skin maturation bioreactors by inducing stress using multiple techniques. However, these bioreactors developed are very complex to build and are not commercially available. As yet, there is no dedicated commercially available bioreactor for the dynamic maturation of skin tissues.

### PRESENTATION OF THE INVENTION

The aim of the invention is therefore to propose a device that makes it possible to obtain cell tissues, for example a tissue that mimics natural skin by having mechanical properties similar to those of natural skin. The invention also aims to propose a device that enables better visual (or microscope-assisted) monitoring of cell development while avoiding manipulations of the culture itself (non-destructively).

To this end, the invention relates to a cell culture device comprising an insert having an internal cavity delimited by an insert side wall and an insert bottom, said internal cavity being able to receive said cell culture in a cell culture fluid (for example a hydrogel) so as to create a cell tissue, said device comprising a container that removably receives said insert, said insert side wall having first through-holes.

The device in accordance with the invention is noteworthy in that said container which accommodates the insert comprises an internal flue, delimiting an internal wall of said container and forming a duct having a first opening at a first duct end and a second opening at a second duct end, opening into a container bottom, said first opening end allowing said insert to be inserted into said flue of the container, said insert bottom comprising second through-holes allowing cells in culture to be observed through the insert bottom via the second opening of the second duct end of said flue, said first and second through-holes being capillary holes able to retain the cell culture fluid in said internal cavity of said insert and serving to anchor said cell tissue.

Indeed, and according to a first technical function enabled by the invention, the through-openings in the form of capillary holes serve to anchor the cell culture fluid in the insert, which makes it possible to obtain a cell tissue that adheres to the wall of the insert, to ensure tension on the tissue to prevent its retraction (the phenomenon of retraction is a phenomenon conventionally observed on cultured tissue, over the course of days when culturing is taking place: the invention avoids this phenomenon).

In addition, according to a second technical function enabled by the invention, the opening in the bottom of the flue wherein the insert is placed makes it possible to see the insert bottom and the capillary through-holes, enabling the cell culture to be observed through the insert bottom without however allowing the cell culture to “leak” through the insert bottom.

“Capillary” means that the holes are small enough to prevent the cell culture fluid, accommodated in the insert cavity, from passing therethrough by virtue of the surface tension of the cell culture fluid.

The invention thus makes it possible to monitor changes in the cell culture during the creation of the cell tissue, and the insert enables the cell culture fluid (and the cell culture developing therein) to be anchored in the first and second through-holes in the insert, so as to enable the cell tissue created to attach to the walls of the insert (side and bottom walls of the insert) in order to create a tissue which is distributed substantially homogeneously in the insert with greater tensions at the anchoring points: this makes it possible to simulate skin anchoring at the edges of a wound, for example (by simulating the organization of a natural extracellular matrix), to maintain the initial surface area of cell tissue created, and also the thickness thereof, and avoids the tissue retraction observed with prior art solutions, as mentioned above.

The device in accordance with the invention may also comprise the following features, taken separately or in combination:

Advantageously, the flue comprises third through-holes, made in the internal wall, for cooperating with said first through-holes when the insert is accommodated in said flue, the third through-holes ensuring the passage of a nutrient fluid between the cavity of the insert and a vessel for receiving the nutrient fluid, said vessel being delimited between a container side wall, the bottom of the container and the internal wall formed by the flue. This allows movement of the nutrient fluid wherein the cell culture fluid (e.g. hydrogel, in the context of an example which will be described and illustrated below) is immersed, which can be accommodated in the device during cell culture, when the device is subjected to movement, in order to recreate stress conditions enabling the cells to respond and equip themselves with mechanical properties close to those of real skin.

According to a non-limiting embodiment described below, the insert side wall comprises at least two rows of first through-holes and the internal flue comprises at least two rows of third through-holes, at least one of said at least two rows of first through-holes cooperating with at least one of said at least two rows of said third through-holes when said insert is inserted into said flue duct.

Even more advantageously, said first through-holes and said second through-holes have substantially the same shape and the same diameter. Preferably, the diameter of the first and second through-holes is between (or substantially between) 1000 and 4000 μm, preferably less than or equal to 2000 μm (or substantially 2000 μm).

According to an advantageous embodiment, the flue comprises first removable fastening elements for the insert, the insert being equipped with second removable fastening elements which are associated with said first removable fastening elements. The insert is thus fastened in the flue by means of removable fastening elements, the insert and the container being equipped with complementary means which, when associated with one another, enable this removable fastening.

The first removable fastening elements for the insert are distributed over at least two levels in said flue, each of said at least two levels being located between said first and second ends of said flue. The insert can thus be positioned, in the context of this embodiment, at two different levels. This thus makes it possible to create different cell development layers in order to reproduce the layers of the skin and to introduce an air/liquid interface during the growth of the tissue (skin), if required.

In another advantageous embodiment, the insert can comprise at least one external lateral protruding lug, said protruding lug comprising at least one blind hole, and the flue can comprise a notch formed in an axial direction through the wall of the flue, the notch having an edge having at least one protruding stud, said at least one blind hole of said at least one protruding lug forming said at least first fastening element for the insert and said at least one protruding stud of said flue forming said at least one second fastening element for the flue, being removably associated with said at least one first fastening element for the insert.

The protruding lug serves here as a removable fastening means. As will be seen below, it can also be used as a gripping element for gripping the insert. Preferably, the insert comprises at least one boss on an edge formed by a free end of said insert side wall, and at least one housing provided in the vicinity of the bottom edge of said insert, said at least one housing having a shape that is complementary to the shape of said at least one boss, said at least one housing being able to receive said at least one boss of an identical second insert positioned beneath the insert of said device. This particular technical feature makes it possible to assemble, by stacking, several inserts of the device on top of one another.

The invention also relates to an insert for a device as defined above. The insert in accordance with the invention comprises an internal cavity delimited by an insert side wall and an insert bottom, said cavity being able to receive said cell culture so as to create a cell tissue, said insert side wall having first through-holes. The insert in accordance with the invention is noteworthy in that the insert bottom comprises second through-holes allowing cells in culture to be observed through the insert bottom, said first and second through-holes being capillary holes able to retain said cell culture fluid in said internal cavity of said insert and being able to serve to anchor the cell tissue.

Preferably, the first and second through-holes have a diameter of between (or substantially between) 1000 and 4000 μm, preferably 2000 μm (or substantially 2000 μm).

In addition, the insert comprises at least one boss on an edge formed by a free end of said insert side wall, and at least one housing provided in the vicinity of the bottom edge of said insert, said at least one housing having a shape that is complementary to the shape of said at least one boss, said at least one housing being able to receive said at least one boss of an identical second insert positioned beneath the insert of said device.

The invention also relates to a facility comprising at least one device as defined above, as well as a support on which said at least one device can be positioned, said support being movably mounted by being associated with a motion-driving apparatus, for driving the motion of the nutrient fluid in which the culture medium is immersed in said insert during formation of said cell tissue.

Finally, the invention relates to a process for producing a cell tissue, using a facility as defined above, said process being noteworthy in that it comprises the following steps:
a) positioning said insert in said flue of said container,
b) positioning said device on said support of said facility,
c) introducing a cell culture fluid and cells to be cultured into the cavity of said insert,
it being possible for step c) to be carried out before step a),
d) introducing a nutrient fluid into said device,
e) actuating the motion-driving apparatus of said support at least partially for a predetermined period of time, to cause motion of said nutrient fluid in said insert, until at least a portion of cell tissue is obtained in said insert,
f) removing said device from said support, and g) extracting said insert from said container, said insert comprising said cell tissue.

Preferably, in the case in which the facility comprises a microscope observation device positioned beneath said support, said support being transparent, the process in accordance with the invention may comprise an additional step d1), between step d) and step e), according to which the development of said cell tissue is observed through said second through-holes of said insert bottom.

Also preferably, in the case in which the device is such that the flue of said accommodating container comprises four studs protruding from the axial edge provided in the wall of the flue, for positioning said insert at four different levels in said flue between the first and the second opening of said flue, the four protruding studs comprising:
a first protruding stud substantially at the level of the bottom of said container, in the vicinity of said second opening, said bottom level of said container defining a first level in said flue,
second and third protruding studs between said second opening and said first opening of the flue, the positioning of the second and third studs defining a second and third level for positioning said insert in the flue of the container,
a fourth protruding stud substantially at the level of the first opening of said flue, the fourth stud defining a fourth level for positioning said insert in the flue, the process has the following particular feature: in step c), said insert is positioned at said first level of said flue, then optionally step d) is carried out or the cells are left to develop for a first predetermined period of time,
at the end of said first predetermined period of time, the insert is positioned at said second level, then step d) is optionally carried out again,
at the end of a second predetermined period of time, the insert is positioned at said third level, then step d) is optionally carried out again.

This step makes it possible to optionally introduce an air/liquid interface: the lower surface and the peripheral edge of the cell culture fluid are in contact with the culture medium (nutrient fluid), while the upper surface of the insert is kept dry for keratinocyte maturation, for example. In one embodiment, which will be described below, the oblong-shaped upper hole is intended to stop the flow of nutrient fluid due to surface tension.

Finally, at the end of a third predetermined period of time, the insert is positioned at said fourth level, then step d) is optionally carried out again, or the cells are left to develop for a fourth predetermined period of time. This step can enable chemical compounds to be tested to be applied to the skin after it has matured.

### DESCRIPTION OF THE FIGURES

Other benefits and features of the invention will become evident upon examining the detailed description of an entirely non-limiting implementation, and from the enclosed drawings wherein:

FIG. 1 is a perspective top view of an insert of a device in accordance with the invention,

FIG. 2 is a perspective bottom view of the insert shown in FIG. 1,

FIG. 3 is a perspective view of several device inserts stacked on top of one another,

FIG. 4 is a perspective top view of a device container in accordance with the invention,

FIG. 5 is a perspective bottom view of the container shown in FIG. 4,

FIG. 6 is a perspective view showing the insert and the container of a device, one above the other, the insert being designed to be inserted into the container,

FIG. 7 is a perspective bottom view of the assembly in accordance with the invention, with the insert being received at a first level in the container,

FIG. 8 is a perspective top view of the assembly shown in FIG. 7,

FIG. 9 is a perspective bottom view of the assembly in accordance with the invention, with the insert being received at a second level in the container,

FIG. 10 is a perspective top view of the assembly shown in FIG. 9,

FIG. 11 is a perspective bottom view of the assembly in accordance with the invention, with the insert being received at a third level in the container,

FIG. 12 is a perspective top view of the assembly shown in FIG. 11,

FIG. 13 is a perspective bottom view of the assembly in accordance with the invention, with the insert being received at a fourth level in the container,

FIG. 14 is a perspective top view of the assembly shown in FIG. 13, and

FIG. 15 is a black-and-white photograph of a skin sample obtained in the first part of the device, and

FIG. 16 schematically shows a facility in accordance with the invention, as well as some steps of a process in accordance with the invention for implementing the facility.

### EMBODIMENT

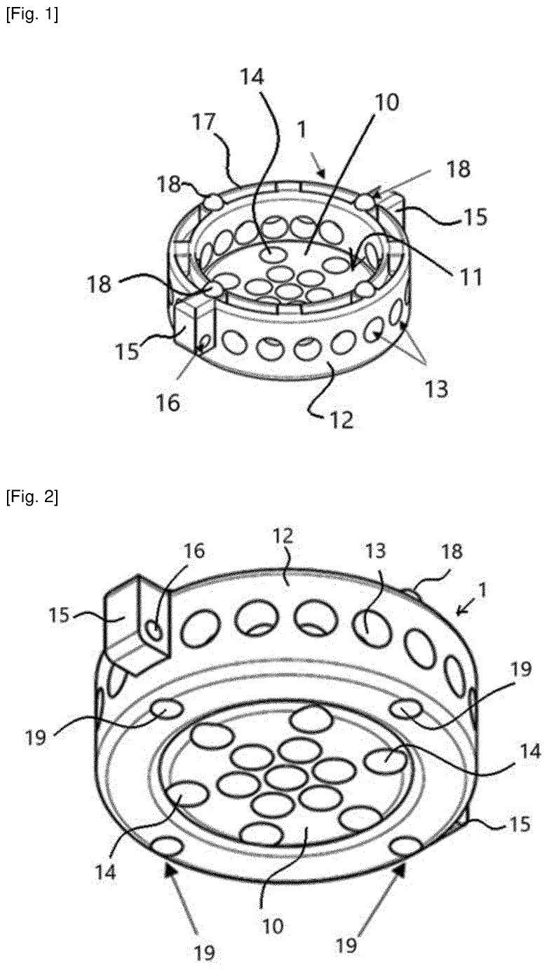

FIGS. 1 to 3 only show an insert 1 of a device in accordance with a first embodiment of the invention.

The function of insert 1 is to accommodate a cell culture and to support the cell tissue being created therein.

In accordance with the invention, the device also comprises a container 3 that removably receives said insert 1: the container 3 will be presented hereafter with reference to FIGS. 4 and 5.

As shown in FIGS. 1 and 2, the insert 1 has the overall shape of a container having a round section and a flat bottom 10.

The insert has an internal cavity 11, which is delimited by an insert side wall 12 and the bottom 10.

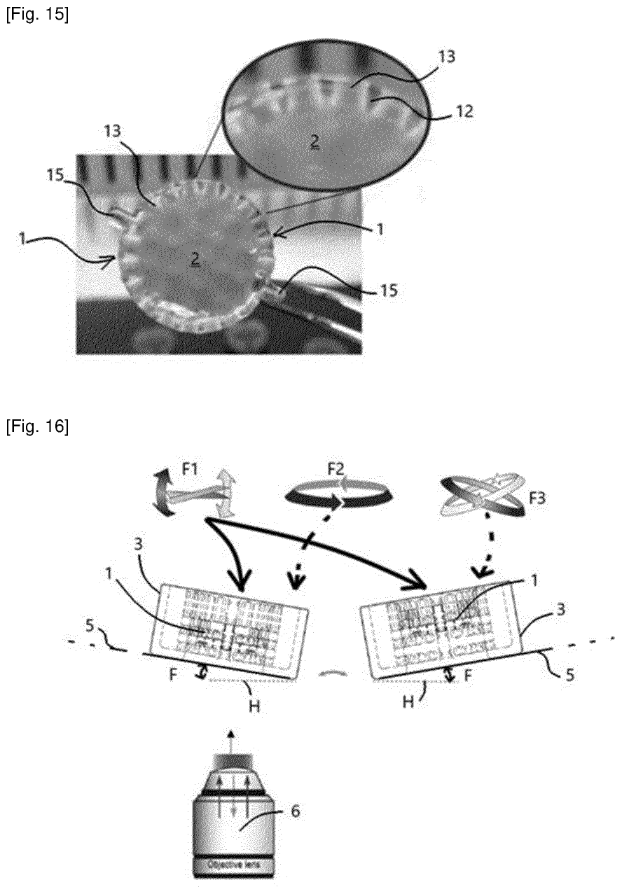

Cell culture is carried out in the cavity 11 of the insert 1, to produce the cell tissue 2, which is shown in particular in FIG. 15.

The side wall 12 of the insert 1 has first through-holes 13, which are small enough to prevent the passage of a cell culture fluid (in the context of this example, this is a hydrogel) wherein cells are able to reproduce, when the cell culture fluid (the hydrogel in the context of the described example) is above a predetermined temperature.

The hydrogel here is the cell culture fluid chosen to feed the cells and maintain the physiological conditions of the medium wherein the cells will develop (e.g. pH, osmolarity, etc.). Serum and antibiotics can be added if necessary.

“Hydrogel” means a gel wherein the swelling agent is water. The matrix of a hydrogel is generally a polymer network. Said polymers are insoluble in water, but are able to swell substantially in the presence of a large quantity of water or aqueous solutions such as biological fluids.

An example of hydrogel may comprise 5% (weight/volume) bovine gelatin, 2% (weight/volume) very-low viscosity alginate (Alpha Aesar #A18565) and 2% (weight/volume) fibrinogen (Sigma #F8630) dissolved in DMEM medium (Gibco™ #21068028) at 37 °C.

In the context of our exemplary embodiment, the hydrogel has:
in the context of a first example of a hydrogel (known by the name Bio ink, the composition of which is indicated in the previous paragraph) a viscosity which may be 1.4×10⁵ Pa·s (or substantially 1.4×10⁵Pa·s) at a temperature of 25 °C, preferably 3.3×10⁻¹ Pa·s (or substantially 3.3×10⁻¹ Pa·s) at 37 °C, and
in the context of a second example of hydrogen, fibrinogen (2% (weight/volume)), a viscosity which may be 4.7×10⁻² Pa·s (or substantially 4.7×10⁻² Pa·s) at a temperature of 25 °C, preferably 1.2×10⁻² Pa·s (or substantially 1.2×10⁻² Pa·s) at 37 °C.

In the context of the invention, the viscosity of cell culture fluids can be greater than or equal to 0.69 mPa·s at 37 °C (viscosity of water).

The diameter of the through-holes 13 is substantially between 1000 and 4000 μm. They are also referred to as “capillary holes”.

At room temperature, the hydrogel has a surface tension such that it enters the through-holes without however passing through them: in this way, the through-holes 13 act as anchoring points for the hydrogel and for the cells developing in the hydrogel.

This allows the cell culture to at least partially enter the through-holes 13 to anchor itself therein, thereby enabling the cell tissue created to be retained on the side wall 12 of the insert 1.

The holes 13 are, in the context of the example shown, evenly distributed around wall 12 so that the cell tissue created can anchor itself evenly right around the wall 12 of the insert 1 and form an overall uniform pellet in the insert which has not retracted in on itself, having regular dimensions in terms of height and thickness (see FIG. 15).

It should be appreciated that the through-holes 13 in the wall 12 of the insert 1 could have a different shape and be more or less numerous without departing from the scope of the invention, or they could be distributed unevenly.

In the context of the illustrated example, the through-holes 13 are distributed mutually equidistantly around the sidewall 12 and form at least a first row of first through-holes.

An insert with a wall 12 having several rows of through-holes 13 could also be provided without departing from the scope of the invention.

FIGS. 1 and 2 also show that the bottom 10 of the insert 1 has second through-holes 14.

These second through-holes 14 allow the cells in culture to be observed through the bottom 10 of the insert 1, for example by means of a device equipped with a camera positioned beneath a transparent support on which the insert, or the container equipped with the insert, rests.

The observer can also view the insert from below in order to observe the cell tissue through the second through-holes 14.

It is envisaged that the second through-holes 14 are also capillary holes which are also able to retain the hydrogel in said cavity 11 and to serve to anchor the cell culture.

The second through-holes preferably have a diameter of between 1000 and 4000 μm. In the context of this example, hole diameters are provided of substantially 2000 μm (or substantially 2000 μm). Thus, the through-holes 13 (hereinafter referred to as first through-holes, to clearly distinguish them from the other through-holes) and the second through-holes 14 serve to anchor said cell tissue, the second through-holes 14 in the bottom 10 of the insert also serving for observation of the development of the cell culture and of the resulting cell tissue.

It is noted in FIGS. 1 to 3 that the insert 1 comprises two lateral lugs 15, protruding from the side wall 12 and extending in a radial direction.

As shown in FIG. 15, these lugs 15 can be used to grip the insert 1, for example with tweezers.

Nevertheless, the lugs 15 have another main function: they enable the insert 1 to be removably positioned in the container 3 (the container 3 will be described below), or even at different levels (or positions) in the container 3. It will be noted that each of the lugs 15 has a transverse blind hole 16, which is also used in the removable fastening of the insert 1 in the container 3, as will be seen below (second fastening element).

FIGS. 1 and 2 also show that the side wall 12 of insert 1 has a free end which forms a notch 17, from which four bosses 18 protrude.

It should be understood that other embodiments could be envisaged without departing from the scope of the invention, and that the insert 1 could have more or fewer bosses 18: for example, a single protruding boss 18, two diametrically opposed protruding bosses 18, or three bosses distributed equidistantly or not, etc.

Positioning means other than bosses and blind holes could be provided: elements having complementary shapes that can be assembled and disassembled could be provided without departing from the scope of the invention.

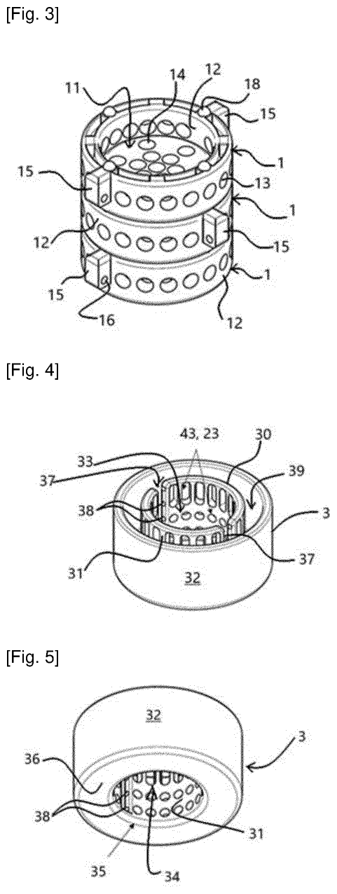

In parallel, in the vicinity of the edge of the insert bottom 10 (see FIG. 2) which joins to the side wall 12, the bottom 10 has four housings 19 having a shape that is complementary to the four bosses 18. Thus, the housings 19 of a first insert 1 can receive the bosses 18 of an identical second insert positioned below the first insert. This makes it possible to stack several inserts on top of one another, so as not to misplace them and so that they can be stored compactly (see FIG. 3). This also makes it possible to carry out several cultures simultaneously, under the same conditions; it is thus possible to culture several identical or non-identical tissues at the same time without bringing them into contact with one another.

It is noted in FIG. 3 that the inserts 1 are stacked with their protruding lugs 15 offset by 90 degrees. Another form of stacking could be envisaged: the lugs 15 could also be aligned on top of one another.

Reference will now be made to FIGS. 4 and 5 to describe the container 3 of the device in accordance with the invention that is able to receive the insert 1.

The container 3 has an overall cylindrical shape.

It comprises an internal flue 30, delimiting an internal wall 31 of said container 3.

In the example shown, the flue 30 is cylindrical in shape.

It should be understood that the shape of the flue 30 could be different, without departing from the scope of the invention. However, the internal shape of the flue is complementary to the external shape, in order to be able to accommodate it.

The container 3 comprises an external side wall 32 which surrounds the internal wall 31 formed by the flue 30.

The internal wall 31 forms a duct in the container 3, the duct having a first opening 33 at a first duct end (FIG. 4) and a second opening 34 at a second duct end (FIG. 5), opening into a bottom 35 of the container 3.

A bottom wall 36 of the container 3 connects the bottom ends of the internal wall 31 and external wall 32.

Thus, the bottom wall 36, internal wall 31, external wall 32 and the bottom wall 36 form a vessel 39 (annular in the context of our example) around the flue.

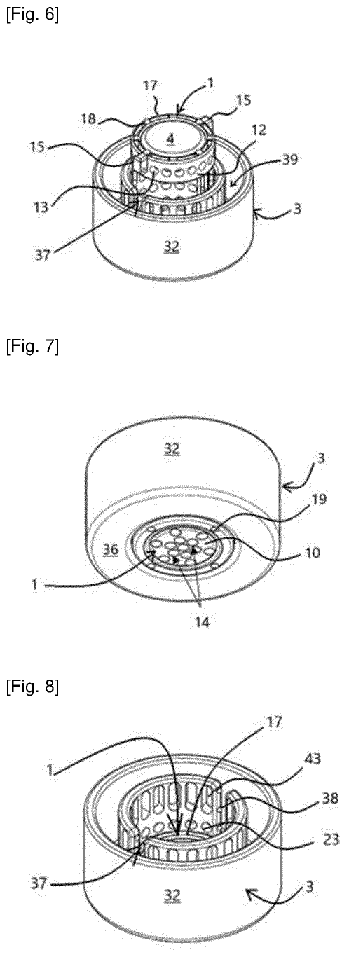

As shown, the flue 30 has internal dimensions that are larger or substantially larger than the external dimensions of the insert 1, and a shape that is complementary to the shape of the insert in order to be able to accommodate it. The first opening 33 allows the insert 1 to be inserted into said flue 30 of the container 3, as shown in FIG. 6. The second opening 34 allows the cell culture to be observed through the insert 1, since the insert 1 bottom is visible through this opening 34.

Reference numeral 4 represents the hydrogel wherein the cell culture is carried out.

Two axial notches 37 are made through the internal wall 31, at least over part of the height of the flue 30, to accommodate the protruding lugs 15 of the insert 1.

It is noted, in particular in FIGS. 4 and 5, that the edges of the notches 37 have four protruding studs 38 which are positioned at four different levels in the notches 37.

The protruding studs 38 ensure that the insert 1 is removably fastened in the flue 30 by insertion of the protruding studs 38 into the blind holes 16 in the lugs 15 of the insert 1.

Thus, the insert 1 can be positioned in the flue 30 at four different levels, for reasons that will be explained below.

The protruding studs 38 are half-dome-shaped and can be deformed to fit into and disengage from the blind holes 16, facilitating changing the position of the insert in the container 3.

It can be seen from FIGS. 4 and 5 that the internal wall 31 of the flue 30 comprises third through-holes 23 and 43, which are distributed over three rows: two rows of through-holes 23, each round in shape, correspond to two levels of positioning of the insert in the container, these two levels being the lowest level (where the insert is positioned at the bottom of the container-see FIGS. 7 and 8) and what is referred to as the “low intermediate” level, located above the low level.

The row of through-holes 43, which each have an axially-oriented oblong shape, extends over two further levels for fastening the insert in the container 3: the high intermediate level, located above the low intermediate level, and the high level.

The third through-holes 23 and 43 are thus formed through the wall of the flue 30 and align with the first through-holes 13 of the insert wall 12 when the insert is positioned in the container, at the low level, low intermediate level, high intermediate level and high level. The alignment of holes 13 and 23, or 13 and 43, allows the nutrient fluid introduced into the vessel 39 to pass through the wall 12 of the insert and through the internal wall 31 of the container, from the cavity 11 to the vessel 39 or from the vessel 39 to the cavity 11, when the insert is in motion and when the viscosity of the hydrogel allows for it: this is possible if the hydrogel is at a temperature below the predetermined temperature mentioned above.

Indeed, below the predetermined temperature (i.e. substantially below a temperature which may be between 15 and 15 °C), the hydrogel has a lower viscosity, enabling it to have a lower surface tension, which enables the hydrogel to pass through the first through-holes 13. The hydrogel does not pass through the through-holes 14 when the insert bottom is positioned on a flat support on which the bottom of the container 36 is also positioned, and when insert 1 is positioned in the container 3 at the low level.

Indeed, in accordance with the process in accordance with the invention, it is provided to set the device in accordance with the invention, and thus the container 3 accommodating the insert 1, in motion while the cells are being cultured. This makes it possible to generate stresses on the cells and to recreate stress conditions enabling the cells to respond and equip themselves with mechanical properties close to those of real skin.

When the device is in motion, the nutrient fluid flows through the first and third through-holes, further promoting anchoring of the tissue in the through-holes 13 of the insert.

For easy passage of the nutrient fluid through the through-holes 13 and 23 or 43, the holes have the same shape and size.

An oblong shape allows more nutrient fluid to pass through. It also allows the hydrogel to pass through several rows of first through-holes 13.

It should be understood, however, that the through-holes 13, 23 and/or 43 may have different shapes and sizes, without however departing from the scope of the invention.

When the insert is positioned in the container 3, it is possible to monitor changes in the generation of the cell tissue without having to remove the insert from the container: indeed, as explained above, the opening 34 (second opening in the flue 30) allows the insert 1 to be viewed from below, and the second through-holes allow the hydrogel and the cell culture to be viewed in the insert cavity.

FIGS. 7 and 8 show the insert 1 positioned at the low level of the flue 30 of the container 3: the through-holes 13 of the side wall 12 of the insert 1 are positioned opposite the row of third holes 23 of the low level. Inserting the lugs 15 of the insert 1 into the notches 37 of the flue 30 imposes an orientation (or position) on the through-holes 13 of the insert in relation to the through-holes 23 or 43 of the flue. In addition, the fact that the lugs 15 are inserted into notches 37 prevents any rotation of the insert 1 relative to the container, ensuring that the first and third through-holes remain correctly aligned.

In this position, where the insert is at the low level in the container 3. This position enables the production of gelled cell cultures.

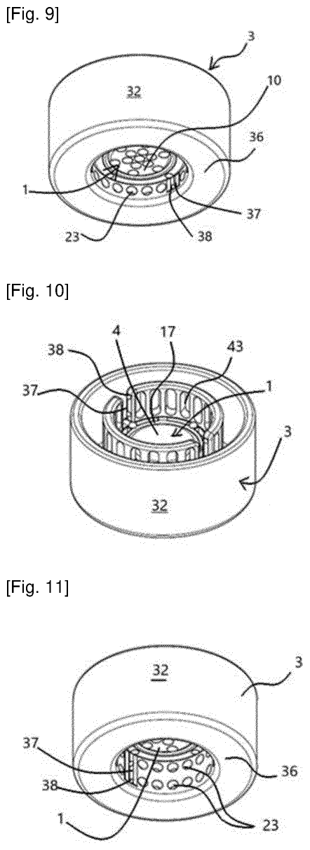

FIGS. 9 and 10 show the insert 1 positioned at the low intermediate level of the flue 30 of the container 3. This position makes it possible to produce immersed cell cultures.

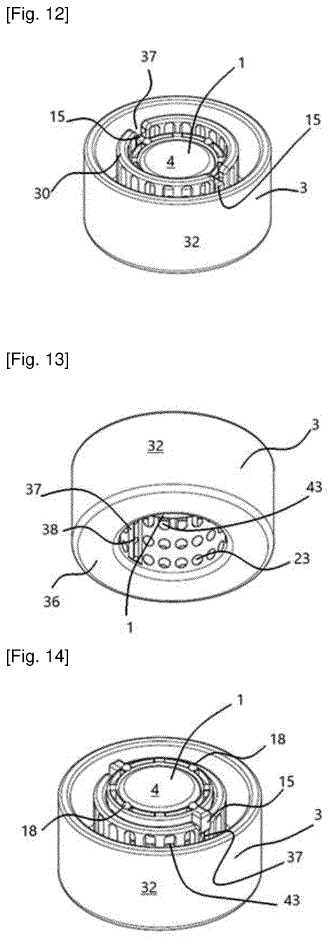

FIGS. 11 and 12 show the insert 1 positioned at the high intermediate level of the flue 30 of the container 3. This position makes it possible to produce cell cultures with air/liquid contact.

FIGS. 13 and 14 show the insert 1 positioned at the high level of the flue 30 of the container 3. This position makes it possible to produce tissues on which cosmetic tests or in vitro drug screening can be carried out, or to study the toxicity of certain products, etc.

Being able to position the insert at several levels (at least two different levels) in order to create different cell development layers makes it possible to reproduce the different layers of the skin and to introduce an air/liquid interface during the growth of the tissue (skin), if required.

FIG. 16 schematically shows a facility in accordance with the invention which comprises at least one device as described above (i.e. comprising at least one insert 1 and a container 3).

FIG. 16 shows two devices, each shown so that the bottom 36 of the container 3 is resting on a flat support 5 which is inclined relative to a horizontal plane H.

Two double arrows F schematically depict that the flat support 5 is driven in motion (it is movable) by a motion-driving apparatus.

The motion-driving apparatus is not actually shown in FIG. 16, but is represented schematically by the double arrows F.

The motions driven by the apparatus are symbolized by the arrows F1, F2 and F3 depicted above the devices in accordance with the invention.

The motion depicted by arrow F1 corresponds to a seesaw motion: the motion-driving apparatus causes the flat support to pivot about a horizontal axis, causing the support 5 to tilt (incline) back and forth relative to the horizontal H, as shown in FIG. 16.

The motion symbolized by arrow F2 is a rotational motion about a vertical axis: the support 5 on which the device in accordance with the invention is positioned can be rotated about its vertical axis.

Finally, the Motion Symbolized by Arrow F3 Is a Motion Combining rotation and seesawing.

By virtue of the motions of the nutrient fluid and of the whole device, physical stress is applied to the cells in culture and stress is generated, leading to developing the cell culture in a way close to that which occurs in reality when cells actually reproduce in a living body.

Driving the motion of the cell culture under development, combined with the positioning of the insert 1 at several levels in the container 3 (enabling different cell culture development conditions), makes it possible to produce cell tissues in the insert that are stronger than those produced by prior art devices. The motion of the cell culture also enables better anchoring of the tissue in the first and second through-holes 13 and 14 described above.

The development of the cell culture in the insert 1 can be observed by virtue of an optical device 6, as is also shown in FIG. 16. The facility can comprise this device, positioned under the transparent flat support 5. The optical device 6 can also be independent and be associated with the facility in accordance with the invention.

Reference will now be made to a process that makes it possible to produce a cell tissue 2, as shown in FIG. 15, by virtue of implementing the facility shown in FIG. 16, comprising at least one device in accordance with the invention.

Firstly, the insert 1 is positioned at the lowest level in the flue 30 of the container 3, and the container 3 is positioned on the flat support 5, so that the bottom 36 of the container and the bottom 10 of the insert 1 are resting on the flat support 5.

Hydrogel is introduced into the insert 1, and cells to be cultured are introduced into the cavity of said insert, into the hydrogel.

It should be noted that, when the hydrogel is introduced into the insert 1, it is at a temperature below the temperature such that it has a viscosity enabling it to pass through the first, second and third through-holes (13, 14, 23 and 43, respectively).

As the process takes place at room temperature, the hydrogel thickens (increase in viscosity), and after a few seconds, the viscosity of the hydrogel is such that it no longer passes through the first, second and third through-holes 13, 14, 23 and 43, allowing it to become anchored therein.

The motion-driving apparatus of the facility is then switched on, and the support on which the device in accordance with the invention is resting is driven into motion for a predetermined period of time. The fluid is then animated by a motion in the insert, generating stress on the hydrogel and the cells in culture therein, for said predetermined period of time: the nutrient fluid crushes the insert and generates a force or stress on the hydrogel containing the cells. This causes the cells inside the hydrogel to mature in order to produce more collagen protein. More generally, this results in inducing either cell differentiation or production of specific extracellular matrix by the cells, or the self-organization of the cells into functional microstructures (microvessels for example). These three phenomena (non-exhaustive) lead to the maturation of a functional tissue. A first layer of cell tissue is thus obtained in the insert 1.

During this first step, the development of the cells can be observed by virtue of the optical device 6, through the second through-holes 14 in the insert bottom.

At the end of the first predetermined period of time, the insert 1 is moved into container 3 to be positioned at the low intermediate level (i.e. just above the low level).

The support is then driven in motion by the motion-driving apparatus (F1, F2 or F3 motion) for a second predetermined period of time.

This operation can be repeated by moving the insert from the low intermediate level to the high intermediate level, or else to the high level, and the support can again be driven in motion (or not) for a predetermined period of time, until the cell tissue obtained meets requirements: for example, certain steps can be repeated, the position of the insert in the container can be changed, the motion of the flat support 5 can be altered, or the motion-driving apparatus can be switched off and the cell tissue can be left to develop within the insert 1 until it has a certain thickness (corresponding, for example, to the depth of the insert 1).

It is thus easy to see how the device in accordance with the invention makes it possible to create cell tissues that are stronger than those obtained by prior art devices, by creating stress conditions during the development phases of the cells, by changing the positioning of the insert 1 in the container, and by creating motions of the hydrogel in insert 1, etc.

By virtue of the presence of capillary holes 13, 14, the cell culture tissue is anchored in the walls of the insert, which makes it possible to obtain a tissue that is close to a real skin cell tissue undergoing reconstitution, for example, pulling on the healthy edges of a wound, for example: indeed, it can be seen in FIG. 15 that the surface of the tissue 2 forms a concavity oriented towards the outside of the insert. It also makes it possible to obtain a tissue which has not retracted in on itself.

As a result, the tissue's properties are even closer to those of the natural tissues that reconstitute themselves after an injury during the healing phase.

It should be understood that the invention is not limited to the embodiment shown in the figures and extends to the implementation of any equivalent means.

The device in accordance with the invention can be made of various materials without departing from the scope of the invention, for example polyacrylate, polycarbonate, polystyrene, ceramic, stainless steel or polyethylene terephthalate.

1-15. (canceled)

16. A cell culture device, comprising: an insert having an internal cavity delimited by an insert side wall and an insert bottom, said internal cavity being able to receive said cell culture in a cell culture fluid so as to create a cell tissue, said device comprising a container that removably receives said insert, said insert side wall having first through-holes, wherein said container comprises an internal flue, delimiting an internal wall of said container and forming a duct having a first opening at a first duct end and a second opening at a second duct end, opening into a container bottom, said first opening end allowing said insert to be inserted into said internal flue of the container,
wherein said insert bottom comprises second through-holes allowing cells in culture to be observed through the insert bottom via the second opening of the second duct end of said flue, said first and second through-holes being capillary holes able to retain said cell culture fluid in said internal cavity of said insert and serving to anchor said cell tissue.

17. The device according to claim 16, wherein said flue comprises third through-holes, made in the internal wall, which cooperate with said first through-holes when the insert is accommodated in said flue, said third through-holes ensuring the passage of a nutrient fluid between the cavity of the insert and a vessel for receiving said nutrient fluid, said vessel being delimited between a side wall of the container, the bottom of the container and the internal wall formed by the flue.

18. The device according to claim 17, wherein said insert side wall comprises at least two rows of first through-holes and wherein the internal flue comprises at least two rows of third through-holes, at least one of said at least two rows of first through-holes cooperating with at least one of said at least two rows of said third through-holes when said insert is inserted into said flue duct.

19. The device according to claim 17, wherein said first through-holes and said second through-holes have substantially the same shape and the same dimensions.

20. The device according to claim 16, wherein said flue comprises first removable fastening elements for the insert, the insert being equipped with second removable fastening elements which are associated with said first removable fastening elements.

21. The device according to claim 20, wherein said first removable fastening elements for the insert are distributed over at least two levels in said flue, each of said at least two levels being located between said first and second ends of said flue.

22. The device according to claim 20, wherein said insert comprises at least one external lateral protruding lug, said protruding lug comprising at least one blind hole,
wherein said flue comprises a notch formed in an axial direction through the wall of the flue, the notch having an edge comprising at least one protruding stud, said at least one blind hole of said at least one protruding lug forming said at least first fastening element for the insert and said at least one protruding stud of said flue forming said at least one second fastening element for the flue, being removably associated with said at least one first fastening element for the insert.

23. The device according to claim 16, wherein said insert comprises at least one boss on an edge formed by a free end of said insert side wall, and at least one housing provided in the vicinity of the bottom edge of said insert, said at least one housing having a shape that is complementary to the shape of said at least one boss, for receiving said at least one boss of an additional and identical second insert positioned beneath the insert of said device.

24. An insert for a device according to claim 16, comprising an internal cavity delimited by an insert side wall and an insert bottom, said cavity being able to receive said cell culture in a cell culture fluid so as to create a cell tissue, said insert side wall having first through-holes, wherein said insert bottom comprises second through-holes allowing cells in culture to be observed through the insert bottom, said first and second through-holes being capillary holes able to retain said cell culture fluid in said internal cavity of said insert and serving to anchor said cell tissue.

25. The insert according to claim 24, wherein said first and second through-holes have a diameter of substantially between 1200 and 2000 μm.

26. The insert according to claim 24, further comprising at least one boss on an edge formed by a free end of said insert side wall, and at least one housing provided in the vicinity of the bottom edge of said insert, said at least one housing having a shape that is complementary to the shape of said at least one boss, for receiving said at least one boss of an additional and identical second insert positioned beneath the bottom of the insert.

27. A facility comprising at least one device according to claim 16, as well as a support on which said at least one device can be positioned, said support being movably mounted by being associated with a motion-driving apparatus, for driving the motion of said cell culture in said insert during formation of said cell tissue.

28. A process for producing a cell tissue, using a facility according to claim 26, said process comprising the following steps:
a) positioning said insert in said flue of said container,
b) positioning said device on said support of said facility,
c) introducing a cell culture fluid and cells to be cultured into the cavity of said insert,
d) actuating the motion-driving apparatus of said support, at least partially for a predetermined period of time, to cause motion of said cell culture fluid in said insert and of the cells in culture in said cell culture fluid, until at least a portion of cell tissue is obtained in said insert,
e) removing said device from said support, and
f) extracting said insert from said container, said insert comprising said cell tissue.

29. The process according to claim 28, said facility comprising an observation device positioned beneath said support, said support being transparent, wherein an additional step d1), between step d) and step e), according to which the development of said cell tissue is observed through said second through-holes of said bottom of the insert.

30. The process according to claim 28, wherein the flue of said accommodating container comprising four studs protruding from the axial edge provided in the wall of the flue, for positioning said insert at four different levels in said flue between the first and the second opening of said flue, the four protruding studs comprising:
a first protruding stud substantially at the level of the bottom of said container, in the vicinity of said second opening, said bottom level of said container defining a first level in said flue,
second and third protruding studs between said second opening and said first opening of the flue, the positioning of the second and third studs defining a second and third level for positioning said insert in the flue of the container,
a fourth protruding stud substantially at the level of the first opening of said flue, the fourth stud defining a fourth level for positioning said insert in the flue,
wherein, in step c), said insert is positioned at said first level of said flue, then optionally step d) is carried out or the cells are left to develop for a first predetermined period of time,
wherein, at the end of said first predetermined period of time, the insert is positioned at said second level, then step d) is optionally carried out again,
wherein, at the end of a second predetermined period of time, the insert is positioned at said third level, then step d) is optionally carried out again, and wherein, at the end of a third predetermined period of time, the insert is positioned at said fourth level, then step d) is optionally carried out again, or the cells are left to develop for a fourth predetermined period of time.
